(19) United States
(12) Patent Application Publication
(10) Pub. No.: US 2009/0062619 A1
(43) Pub. Date: Mar. 5, 2009

(54) METHODS AND APPARATUS FOR SURGICAL RETRACTION

(76) Inventors: Todd M. Bjork, River Falls, WI (US); Dan McPhillips, Ham Lake, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 12/056,044

(22) Filed: Mar. 26, 2008

Related U.S. Application Data
(60) Provisional application No. 60/920,000, filed on Mar. 26, 2007.

(51) Int. Cl. A61B 1/32

(52) U.S. Cl. 600/219

(57) ABSTRACT

The present invention provides method and apparatus that may be patient mounted for surgical retraction. The retractor disclosed herein may be positioned with a base plate resting against the patient's skin surface. In one embodiment, retractor blades extending into the patient's body from the base plate of the retractor device may be adapted to be inserted in a closed position for minimally invasive access, and adapted to be moved away from each other in a controlled fashion to create the desired surgical retraction of soft tissue. Because the blades are inserted in a closed position, the skin incision is small compared to the available exposure of nerves and delicate tissue.

### RELATED APPLICATION

This application claims the benefit of U.S. Provisional Application No. 60/920,000, filed Mar. 26, 2007, which is hereby fully incorporated herein by reference.

### FIELD OF THE INVENTION

The present invention relates generally to a method and apparatus for retracting tissue in a surgical procedure. More particularly, the present invention relates to a surgical retractor system adapted to be patient mounted and/or utilizes two or more blades to create and maintain a minimally invasive patient access.

### BACKGROUND OF THE INVENTION

Surgical retractors are commonly used to move a patient's tissue apart in order to provide a surgeon with an operating field. There exist a myriad of surgical retractors. Most are manual instruments held in place by the surgeon or a surgical assistant. There also are retractors that are mounted on the operating table via some form of a frame system and a post device that mounts to the rails on the operating table. Table-mounted systems are advantageous in maintaining the desired retraction and exposure while freeing the hands of the surgical team members for other tasks. For example, U.S. Pat. No. 7,156,805 to Thalgott et al. discloses a retractor system having an adjustable blade holder, where the retractor system is mounted to an operating table. Unfortunately, table mounting requires a frame assembly that can be time consuming and complex to set up. Further, generally the table mounting assemblies are generally large and can obstruct the surgical team's access to the patient.

Not all retractors are table mounted, for example, U.S. Pat. No. 5,027,793 to Engelhardt et al describes a retractor with spikes to engage bone. The device of the '793 patent is engaged into bone adjacent the surgical site and removed after the procedure is finished. Because the entire assembly is engaged to the bone, the device required a relatively large access opening and is limited in its use to those procedures adjacent to a bone structure that is large enough and sturdy enough to support the retractor frame.

U.S. Pat. No. 7,001,397 to Davison et al. discloses a retractor for use in endoscopic surgery. The retractor disclosed in the '397 patent employs a conical distal end portion that is deployed at the surgical site. Upon its expansion, the conical portion retracts the tissue apart at the surgical site creating an operating field. However, the device disclosed in the '397 Patent must either be held by a surgical assistant or linked to a table mounted frame during use.

In addition to the creation of an operating field, many surgical procedures require several instruments for implanting devices or removing tissue or other material. These instruments may also conventionally need to be mounted to the table or held in a specific position during use.

There is a need for a retractor device that can be patient mounted, that is stable and can establish and maintain a surgical operating field and access thereto for surgical instruments and other devices.

### SUMMARY OF THE INVENTION

The present invention provides method and apparatus that may be patient mounted for surgical retraction. The retractor disclosed herein may be positioned with a base plate resting against the patient's skin surface. In one embodiment, retractor blades extending into the patient's body from the base plate of the retractor device may be adapted to be inserted in a closed position for minimally invasive access, and adapted to be moved away from each other in a controlled fashion to create the desired surgical retraction of soft tissue. Because the blades are inserted in a closed position, the skin incision is small compared to the available exposure of nerves and delicate tissue.

According to one aspect of the present invention, articulation of the blades at the surgical site creates a locking hold in the incision area such that the device is self-supporting.

According to another aspect of the present invention, mounting of the retractor may be augmented by the use of a table or frame mount if a particular surgical access angle is desired.

In one embodiment, the retractor may include attachments to hold, support and guide other non-retractor instruments and/or components.

In another aspect of the retractor adapted for spinal surgeons, the retractor is minimally invasive yet creates an opening sufficient for a surgeon to reach at least two levels of vertebrae in one incision. In yet another aspect, the retractor while being minimally invasive creates a surgical access opening large enough to allow a surgeon to place at least three levels of pedicle screws through one incision.

In an embodiment of the present invention, each side of the retractor may be adjusted independently of the other. According to one aspect, the blades may retract and articulate simultaneously. In one embodiment, the articulation and retraction occur in a direct relationship, such that the more retraction occurs the more articulation will occur. In this embodiment, increased articulation with increasing retraction enhances the self-locking stability of the device as surgical exposure is increased.

In one embodiment of the present invention, the ramp angle may be adjusted such that the angle of the travel path for each blade is between about 0 degrees and 75 degrees.

In an embodiment of the present invention, the blades may be connected to the frame of the retractor by locking the blades under a tab. While locked under the tab, the blades may be swung to the midline of the retractor frame and then the blades may be locked under a spring loaded catch which holds the blades in position. In one embodiment, the blades may be removed by turning or pushing the catch to an unlocked position and swinging the blade out.

According to one aspect of the present invention, the retractor may form a base or platform to maintain the position of an access portal that can be used to deliver or remove tissue, bone, implants or other items necessary for a particular surgical procedure. The position of the access portal may be maintained while simultaneously maintaining visualization of the anatomy and maintaining the access trajectory.

In an embodiment, the retractor blades may be interchangeable. According to one aspect, the blade size may be chosen relative to the drilling dilator used. Various lengths of the blades may used to adapt to a patient's anatomy. According to one aspect, the retractor may include at least 2 blades.

In one embodiment of the present invention, the retractor may be comprised of a plastic or any suitable radio-opaque or radio-lucent material for optimal imaging. In another embodiment, the blades may be comprised of a material different than the base plate.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of a retractor of the present invention.

FIG. 2 is a perspective view of a blade according to an embodiment of the present invention.

FIG. 3 is a perspective view of a frame according to an embodiment of the present invention.

### DETAILED DESCRIPTION

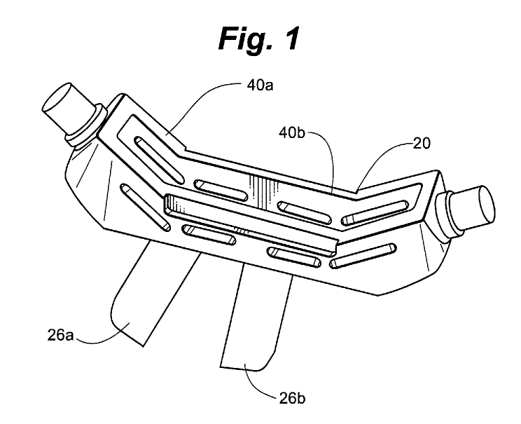

Referring to FIG. 1, there can be seen a retractor according to one embodiment of the present disclosure. In the embodiment shown in FIG. 1, retractor 10 includes a frame 20 and a removable blade set having blades 26a and 26b. It is noted that the retractor may include more than two blades. For example, it may be desirable to retract in a transverse plane. In such a case the retractor may include more than two blades and correspondingly more than two blade holders.

In one embodiment, blades 26a and 26b may be selected from a variety of blade lengths ranging from about 25 mm to about 100 mm. The blade length may be selected such that the blades reach the desired depth required by a particular patient's anatomy and the particular surgical procedure being performed. According to one aspect, blades 26a and 26b may include driving edges that may be tapered to fit closely over a dilator. In an embodiment, the driving edges may enable the blades 26a and 26b to twist down into position at the surgical site.

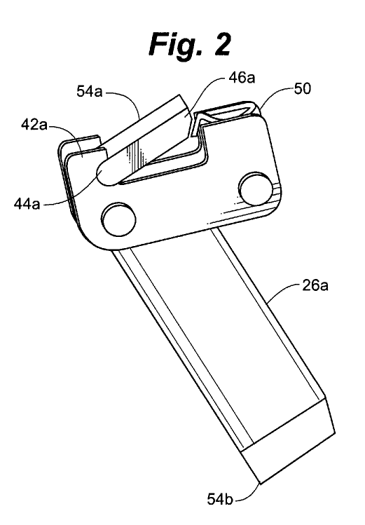

In one embodiment, frame 20 includes blade holders 40a and 40b. The blades 26a and 26b may be operably engaged to the blade holder. Referring to FIG. 2, a representative blade 26a may be seen. Blade 26b would have corresponding components and function. Blade 26a has a proximal end 54a and a distal end 56a. The proximal end 54a includes a tab engaging portion 44a and a catch engaging portion 46a. The tab engaging portion 44a may slide under a tab 42a in the blade holders 40a. Once blade 26 is locked under tab 42a, blade 26a may be swung toward the midline of the frame 20 such that catch engaging portion 46a are placed under a spring loaded ear or catch 50.

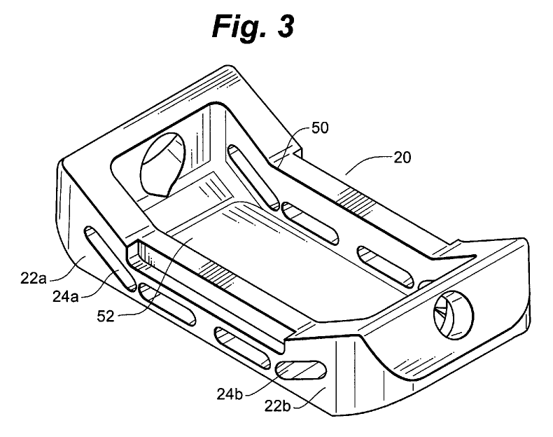

Referring now to FIG. 3, frame 20 may include ramp portions 22a and 22b which each include a travel path 24a and 24b for blades 26a and 26b respectively. The ramp portions 22a and 22b may be adjustable such that the angle of the travel paths 24a and 24b is between about 0 degrees and 75 degrees. The length of frame 20 may be adjusted such that blades 26a and 26b may be farther apart along the axis of frame 20. The angled travel paths 24a and 24b may provide for articulation of the blades 26a and 26b. The blades 26a and 26b may travel along the travel paths 24a and 24b in linear and angular motion.

In one embodiment, frame 20 includes blade holders 28a and 28b. The blade holders 28a and 28b may each include a linear cam and an angular cam respectively. The cams may engage cam followers in ramps 22a and 22b respectively. Once engaged, the blades 26a and 26b may each be moved individually in a combined linear/angular motion. Each blade may be independently adjusted. The travel paths in frame 20 engage may engage the blade holders 28a and 28b such that translational and rotational motion is combined in one step, in that angulation occurs with translation.

Frame 20 includes a predetermined upper opening 50. The upper opening 50 may be in the range of about 6 mm to about 30 mm in length. The lower opening 52 is also predetermined in that the blades 26a and 26b are engaged to the blade holders 28a and 28b in predetermined travel paths. The length of each blade and the travel path of its respective blade holder determine the opening of the retracted area at the distal end of the blades. For a given travel path, a longer blade will result in a broader opening. The lower opening 52 may be in the range of about 6 mm to about 100 mm in length.

In one preferred embodiment, the blades 26a and 26b may open a surgical exposure or working area at their distal end moving in an arc creating the opening in the cephalo-caudal plane as opposed to many conventional retractors which create a large symmetrical (circular) opening, that is as wide in the transverse medial-lateral plane as it is in the cephalo-caudal plane. Creating an opening in the cephalo-caudal plane may be particularly suited to minimally invasive multiple-level spinal procedures.

The blades 26a and 26b may be finger blades. In one embodiment the blades 26a and 26b may be rounded, in another embodiment, the blades 26a and 26b may be flat or almost flat. In one preferred embodiment the blades 26a and 26b may be semi-cylindrical such that the blades may be inserted into the patient's body in a tube-like configuration fitting over a tissue dilator.

In an embodiment, the blades 26a and 26b may be made of nitinol with an arc extending along the side of the blade to prevent tissue from pushing in over the side of the blade and interfering with the retracted surgical field. In another embodiment, fanned blades may be inserted alongside the retracting blades 26a and 26b, to establish a “curtain” that holds back the soft tissue along the long sides of the retracted opening.

According to one embodiment, the blades may be moved individually in an incremental manner using a screw, knob, ratcheting lever or other mechanism integral with or detachably mounted to the frame. As the blades are opened, moving in a translational and angular path, the tissue is distracted and pushed up toward the patient's skin surface and may become entrapped between the blades 26a and 26b and the patient's body. Entrapping the tissue in this manner compresses the tissue more gently, creating a locally more rigid tissue platform which further secures the frame 20 on the patient's body.

The retractor disclosed herein may be used in any surgical procedure requiring a retracted surgical access opening. By way of example, the retractor and a method of its use will be described herein with respect to spine surgery. In an embodiment the retractor may be used to create a surgical access opening large enough to allow access to at least two vertebral disc places and three levels of pedicles, in order to accommodate a one or two-level interbody fusion procedure. The retractor may be used with or without introductory dilators. By way of example, in an interbody fusion procedure, an incision is created just large enough to place a small guide pin or dilator, in the range of about 2 mm to about 8 mm. The first dilator is placed into the vertebral pedicle or the annulus of the intervertebral disc in its desired position. Then progressively larger dilators are placed over the first dilator until the desired dilation is accomplished. In an embodiment, the dilation proceeds until the opening is at least as large as the inside diameter of the blades, in the range of about 6 mm to about 20 mm. The dilators may vary in length and may include depth markings to assist in determining the length of the blades to be used in order to reach the bony structure of the spine.

Once the desired blades are selected, the blades may be placed into the retractor frame assembly as described hereinabove. The retractor, in the closed position, may then be placed over the dilators and slid into place such that the base of the retractor frame lies flat on the patient's skin. The dilators may then be removed. The retractor may then be opened to create the desired surgical access opening. As the retractor is opened, the blades move apart along a predetermined path. Each of the blades is independently adjustable as described herein above. Once the desired opening is created, a dilator pin may be placed through the opening into the disc space at the desired angle. A support system, such as a table-mounted frame apparatus may be locked to the retractor frame if desired.

The surgeon will then determine the desired access trajectory and then an access portal may be placed over the dilator pin and through the retractor frame and into the disc space. The access portal may be adapted for mounting to the retractor frame for greater stability and trajectory control, and to dissipate impact or compressive forces which may be applied to instruments used during the surgical procedure, as disclosed in U.S. patent application Ser. No. 11/655,730. The surgeon may then proceed with the fusion procedure. In an embodiment, an Optimesh® container may be implanted into the prepared disc space and filled with fill material to distract the vertebrae, restoring the desired disc space height and stabilizing the motion segment as well as promoting fusion. Once the procedure is completed, the blades may be returned to a closed position and the retractor can be removed.

Various modifications to the disclosed apparatuses and methods may be apparent to one of skill in the art upon reading this disclosure. The above is not contemplated to limit the scope of the present invention, which is limited only by the claims below.

1. A patient mounted surgical retractor comprising:
a frame having a patient contacting surface, the frame having a first blade holder and a second blade holder;
a first blade operably engaged to the first blade holder and a second blade operably engaged to the second blade holder;
the frame configured to provide independent movement of the first blade holder and the second blade holder from a first closed position to a second fully retracted position; and
an activation mechanism that independently moves the first blade and the second blade from the first closed position to the second fully retracted position.

2. The retractor of claim 1 wherein the blades are configured to move linearly.

3. The retractor of claim 1 wherein the blades are configured to move angularly.

4. The retractor of claim 1 wherein the blades are configured for simultaneous linear and angular motion.

5. The retractor of claim 1 wherein the frame includes a first travel path and a second travel path, the first and second travel paths adapted for operable engagement with the first and second blade holders.

6. The retractor of claim 5 wherein the first travel path and the second travel path are configured to facilitate simultaneous translational and rotational motion of the first and second blades.

7. The retractor of claim 5 wherein the first travel path and the second travel path are configured to facilitate independent translational motion of the first and second blades.

8. The retractor of claim 5 wherein the first travel path and the second travel path are configured to facilitate independent rotational motion of the first and second blades.

9. The retractor of claim 5 wherein the first travel path and the second travel path are configured to facilitate independent translational and rotational motion of the first and second blades.

10. The retractor of claim 1 wherein the activation mechanism moves the first and second blades incrementally to any position between the first closed position and the second fully retracted position.

11. The retractor of claim 1 including a proximal opening and a distal opening.

12. The retractor of claim 11 wherein the proximal opening may be in the range of about 6 mm to 30 mm in length.

13. The retractor of claim 11 wherein the distal opening may be in the range of about 6 mm to about 100 mm in length.

14. A method of patient mounted surgical retraction comprising:
creating a surgical access opening;
placing a retractor having a set of blades in a closed position into the surgical opening such that a patient contacting surface of the retractor lies generally flat on the patient;
activating the retractor such that the blades move apart upon a predetermined path in a simultaneous linear and angular motion creating a distal opening larger than the surgical access opening.

15. A method of patient mounted surgical retraction comprising:
providing a retractor having a patient contacting surface and a set of blades;
providing instructions for using the retractor including the steps of:
creating a surgical access opening;
placing the retractor with the blade set in a closed position into the surgical opening such that the patient contacting surface of the retractor lies generally flat on the patient;
activating the retractor such that the blades move apart upon a predetermined path in a simultaneous linear and angular motion creating a distal opening larger than the surgical access opening.

16. A method of patient mounted surgical retraction comprising:
creating a surgical access opening;
placing a retractor having a blade set in a closed position into the surgical opening such that a patient contacting surface of the retractor lies generally flat on the patient;
activating the retractor such that the blades move apart independently of each other upon a predetermined path in a linear and/or angular motion creating a distal opening larger than the surgical access opening.

17. A method of patient mounted surgical retraction comprising:
providing a retractor having a patient contacting surface and a set of blades;
providing instructions for using the retractor including the steps of:
creating a surgical access opening;
placing a retractor having the blade set in a closed position into the surgical opening such that a patient contacting surface of the retractor lies generally flat on the patient;
activating the retractor such that blades move apart independently for each other upon a predetermined path in a linear and/or angular motion creating a distal opening larger than the surgical access opening.
